(19) United States
(12) Patent Application Publication
(10) Pub. No.: US 2017/0333459 A1
(43) Pub. Date: Nov. 23, 2017

(54) METHODS OF TREATING ORTHOPOX VIRUS INFECTIONS AND ASSOCIATED DISEASES

(71) Applicant: Chimerix, Inc., Durham, NC (US)

(72) Inventors: Ernest Randall Lanier, Chapel Hill, NC (US); George R. Painter, Atlanta, GA (US)

(21) Appl. No.: 15/669,084

(22) Filed: Aug. 4, 2017

Related U.S. Application Data
(63) Continuation of application No. 13/092,611, filed on Apr. 22, 2011.
(60) Provisional application No. 61/326,994, filed on Apr. 22, 2010.
(60) Provisional application No. 61/327,919, filed on Apr. 26, 2010.
(60) Provisional application No. 61/328,491, filed on Apr. 27, 2010.
(60) Provisional application No. 61/333,607, filed on May 11, 2010.
(60) Provisional application No. 61/413,079, filed on Nov. 12, 2010.

(51) Int. Cl. A61K 31/675

(52) U.S. Cl. CPC A61K 31/675

(57) ABSTRACT

The present invention provides methods of treating diseases associated with at least one virus. The methods include administering a compound described in the invention in a therapeutically effective amount. According to some aspects of the present invention, the methods provide treatment of an orthopox virus infection or a disease related to orthopox virus.

### RELATED APPLICATIONS

This application is a continuation of U.S. Non-Provisional Patent application Ser. No. 13/092,611, filed Apr. 22, 2011, now pending, which claims the benefit under 35 U.S.C. §119(e) of U.S. Provisional Patent Application No. 61/326,994, filed Apr. 22, 2010; U.S. Provisional Patent Application No. 61/327,919, filed Apr. 26, 2010; U.S. Provisional Patent Application No. 61/328,491, filed Apr. 27, 2010; U.S. Provisional Patent Application No. 61/333,607, filed May 11, 2010; and U.S. Provisional Patent Application No. 61/413,079, filed Nov. 12, 2010; the disclosures of which are incorporated herein by reference in their entireties.

### GOVERNMENT LICENSE RIGHTS

This invention was made with government support under Grant No. 5U01AI057233 awarded by the National Institutes of Health. The government has certain rights in the invention.

### FIELD OF THE INVENTION

The present invention concerns methods of treating diseases associated with at least one orthopox virus with a prodrug of cidofovir.

### BACKGROUND OF THE INVENTION

Cidofovir is taken up by pinocytosis and requires intravenous infusion that can result in nephrotoxicity. The lipid analogue, hexadecyloxypropyl-cidofovir (CMX001), is orally bioavailable and no nephrotoxicity has been detected in preclinical toxicity studies or human trials. CMX001 is under development as an active IND drug (Kern, Hartline et al. 2002; Keith, Wan et al. 2004; Painter and Hostetler 2004; Beadle, Wan et al. 2006; Lebeau, Andrei et al. 2006). CMX001 (1-O-hexadecyloxypropyl cidofovir, HDP-cidofovir) is a lipid conjugate of cidofovir. Mechanistically, the lipid moiety dictates the drug's pharmacokinetic properties in target organs, while the antiviral activity is contained within the nucleotide residue. Compared to cidofovir, which is taken up into cells by inefficient processes, the conjugate is designed to act like lysophosphatidylcholine (LPC) utilizing natural lipid uptake pathways to achieve high intracellular concentrations. Once inside target cells, the lipid side chain of CMX001 is cleaved, presumably by phospholipase C, to yield free cidofovir. Conversion of cidofovir to the active antiviral agent, cidofovir-PP (cidofovir diphosphate), occurs via a two-step phosphorylation process catalyzed by intracellular anabolic kinases. Cidofovir-PP exerts its antiviral effects intracellularly by acting as a potent alternate substrate inhibitor of viral DNA synthesis.

Orthopox viruses include, but are not limited to, variola, vaccinia, monkeypox, and molluscum contagiosum viruses. Variola causes the deadly disease smallpox. There is increased concern about smallpox as a bioterrorism agent. Monkeypox causes disease in primates and other animals and occasionally causes disease in humans. Purposeful inoculation with live vaccinia can lead to mild, transitory infection. The immune memory provoked by vaccinia infection then either prevents smallpox infection from occurring, or renders smallpox infection harmless. Inoculation with strains of vaccinia can have toxic side effects in some persons, creating a need for safer alternative vaccines. Further, the administration of vaccines to those with weakened immune systems (e.g., due to HIV infection, immunosuppressive drug therapy for organ transplantation, or chemotherapy for cancer treatment) or other conditions (e.g., pregnant, eczema, atopic dermatitis) can be problematic. Thus, other treatment and/or prevention options for orthopox virus infections are needed.

### SUMMARY OF THE INVENTION

A first aspect of the invention is methods of treating conditions/disease associated with at least one virus in a subject. The method comprises administering to the subject a therapeutically effective amount of compounds described herein. The compounds described herein are specifically targeted against viral replication and/or virally infected/transformed cells. In one embodiment, the subject is immunocompromised.

In one embodiment, the viral infection or disease is associated with orthopox virus.

In some embodiments, the disease associated with orthopox virus is selected from variola major and minor, vaccinia, smallpox, cowpox, camelpox, mousepox, rabbitpox, monkeypox, and molluscum contagiosum.

Preferably the compound of the invention is administered orally, preferably at a dosage of from about 1 mg/kg to about 100 mg/kg, more preferably at a dosage of from about 1 mg/kg to about 20 mg/kg. For example, said compound is administered to said subject at a dosage of 1, 2, 3, 4, 5, 6, 7, 8, 9, 10, 11, 12, 13, 14, 15, 16, 17, 18, 19 or 20 mg/kg. In addition, said compound is administered to said subject in an amount of 10, 25, 50, 75, 100, 125, 150, 175 or 200 mg. The compounds of the invention can be administered, for example, as a single dose, daily, twice daily, or every other day.

In one embodiment, the compound which is orally administered is:

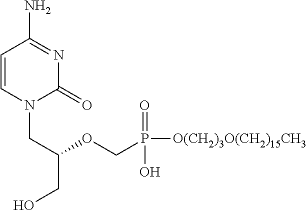

or a pharmaceutically acceptable salt thereof.

With respect to disorders associated with viral infections, the “effective amount” is determined with reference to the recommended dosages of the antiviral compound. The selected dosage will vary depending on the activity of the selected compound, the route of administration, the severity of the condition being treated, and the condition and prior medical history of the patient being treated. However, it is within the skill of the art to start doses of the compound(s) at levels lower than required to achieve the desired therapeutic effect and to gradually increase the dosage until the desired effect is achieved. If desired, the effective daily dose may be divided into multiple doses for purposes of administration, for example, two to four doses per day. It will be understood, however, that the specific dose level for any particular patient will depend on a variety of factors, including the body weight, general health, diet, time, and route of administration and combination with other drugs, and the severity of the disease being treated.

The compounds of the invention can be administered, for example, once per day for 1, 2, 3, 4, 5, 6, 7, 8, 9, or 10 days or more. For example, 1 mg/kg can be administered once per day for 5 days. For example, 5 mg/kg can be administered once per day for 5 days. For example, 10 mg/kg can be administered once per day for 5 days. For example, 15 mg/kg can be administered once per day for 5 days. For example, 20 mg/kg can be administered once per day for 5 days.

For example, a single dose of 20 mg/kg can be administered. For example, a single dose of 30 mg/kg can be administered. For example, a single dose of 50 mg/kg can be administered. For example, a single dose of 75 mg/kg can be administered. For example, a single dose of 100 mg/kg can be administered.

The compounds of the invention can be administered, for example, twice per day for 1, 2, 3, 4, 5, 6, 7, 8, 9, or 10 days or more. For example, 1 mg/kg can be administered twice per day for 5 days. For example, 5 mg/kg can be administered twice per day for 5 days. For example, 10 mg/kg can be administered twice per day for 5 days. For example, 15 mg/kg can be administered twice per day for 5 days. For example, 20 mg/kg can be administered twice per day for 5 days. For example, three doses of 20 mg/kg of a compound of the invention can be administered every other day.

For example, a first dose of a compound of the invention is administered in an amount of 200 mg, followed by subsequent doses administered in an amount of 100 mg. For example, a first dose of a compound of the invention is administered in an amount of 200 mg, followed by a second dose administered 6 days after the first dose in an amount of 100 mg, followed by subsequent doses administered in an amount of 100 mg every 6 days.

The compounds of the invention can be administered, for example, once or twice per week. For example, administration of the compound can be for 1, 2, 3, 4, 5, 6, 7, 8, 9, or 10 weeks or more. For example, about 4 mg/kg can be administered once per week. For example, about 4 mg/kg can be administered twice per week. For example, 100 mg can be administered once per week. For example, 200 mg can be administered once per week. For example, 300 mg can be administered once per week. For example, 100 mg can be administered twice per week. For example, 200 mg can be administered twice per week. For example, 300 mg can be administered twice per week.

A further aspect of the invention provides methods for treating disease associated with at least one virus in a subject in need of an immunosuppressant agent. The methods include administering to the subject a therapeutically effective amount of compound described herein in combination with one or more immunosuppressant agents.

In some embodiments, at least one immunosuppressant agent is selected from Daclizumab, Basiliximab, Tacrolimus, Sirolimus, Mycophenolate (as sodium or mofetil), Cyclosporine A, Glucocorticoids, Anti-CD3 monoclonal antibodies (OKT3), Antithymocyte globulin (ATG), Anti [symbol] CD52 monoclonal antibodies (campath 1-H), Azathioprine, Everolimus, Dactinomycin, Cyclophosphamide, Platinum, Nitrosurea, Methotrexate, Azathioprine, Mercaptopurine, Muromonab, IFN gamma, Infliximab, Etanercept, Adalimumab, Tysabri (Natalizumab), Fingolimodm or a combination thereof.

In one embodiment, at least one immunosuppressant agents is Tysabri (natalizumab).

### DETAILED DESCRIPTION OF THE INVENTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this invention belongs.

All patents, patent applications and publications referred to herein are incorporated by reference in their entirety. In case of a conflict in terminology, the present specification is controlling.

Subjects to be treated by the methods of the present invention are, in general, mammalian and primate subjects (e.g., human, monkey, ape, chimpanzee). Subjects may be male or female and may be of any age, including prenatal (i.e., in utero), neonatal, infant, juvenile, adolescent, adult, and geriatric subjects. Thus, in some cases the subjects may be pregnant female subjects.

As used herein, or “a therapeutically effective amount” refers to an amount that will provide some alleviation, mitigation, and/or decrease in at least one clinical symptom in the subject. Those skilled in the art will appreciate that the therapeutic effects need not be complete or curative, as long as some benefit is provided to the subject.

As used herein, “specificity” or “specifically against” refers to a compound that may selectively inhibit the metabolic activity and/or DNA replication of a certain type of virally infected cells. The specificity may be tested by using any methods known to one skilled in the art, for example, testing IC₉₀ and/or IC₅₀. In some embodiments, the compounds described herein may have IC₉₀ and/or IC₅₀ against viral infected cells to be at least about three fold lower than the IC₉₀ and/or IC₅₀ against normal (uninfected) cells. In some embodiments, the compounds described herein may have IC₉₀ and/or IC₅₀ against viral infected cells to be about three fold to ten fold lower than the IC₉₀ and/or IC₅₀ against normal (uninfected) cells. In some embodiments, the compounds described herein may have IC₉₀ and/or IC₅₀ against viral infected cells to be at least ten fold lower than the IC₉₀ and/or IC₅₀ against normal (uninfected) cells. In some embodiments, the compounds described herein may have specific cytotoxicity against viral infected and/or transformed cells. The cytotoxicity may be measured by any methods known to one skilled in the art.

Unless otherwise stated, structures depicted herein are meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

As used herein, the terms “treatment,” “treat,” and “treating” refer to reversing, alleviating, inhibiting the progress of a disease or disorder as described herein, or delaying, eliminating or reducing the incidence or onset of a disorder or disease as described herein, as compared to that which would occur in the absence of the measure taken. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Active compounds of the present invention may optionally be administered in combination (or in conjunction) with other active compounds and/or agents useful in the treatment of viral infections as described herein. The administration of two or more compounds “in combination” or “in conjunction” means that the two compounds are administered closely enough in time to have a combined effect, for example an additive and/or synergistic effect. The two compounds may be administered simultaneously (concurrently) or sequentially or it may be two or more events occurring within a short time period before or after each other. Simultaneous administration may be carried out by mixing the compounds prior to administration, or by administering the compounds at the same point in time but at different anatomic sites or using different routes of administration. In some embodiments, the other antiviral agent may optionally be administered concurrently.

“Parenteral” as used herein refers to subcutaneous, intravenous, intra-arterial, intramuscular or intravitreal injection, or infusion techniques.

“Topically” as used herein encompasses administration rectally and by inhalation spray, as well as the more common routes of the skin and mucous membranes of the mouth and nose and in toothpaste.

According to some aspects of the present invention, compounds with a range of biological properties are provided. Compounds described herein have biological activities relevant for the treatment of diseases associated with at least one virus.

According to one aspect of the present invention, the compounds have the structure of Formula A, or A′:

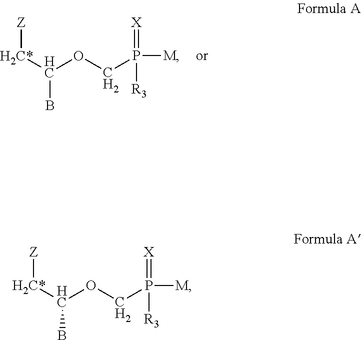

wherein:

M is

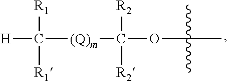

and the oxygen of M is bonded to —P(═X)(R₃)—,

Q, when present, is:

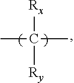

R₁, R₁′, R₂, R₂′, Rₓ and R_{y} are independently —H, halogen, —ORⁱ, —SRⁱ, —NHRⁱ, or —NRⁱRⁱⁱ, and Rⁱ and Rⁱⁱ are independently hydrogen or an aliphatic moiety,

and m is an integer from 0 to 6,

B is selected from the group consisting of hydrogen, F, CF₃, CHF₂, —CH₃, —CH₂CH₃, —CH₂OH, —CH₂CH₂OH, —CH(OH)CH₃, —CH₂F, —CH═CH₂, and —CH₂N₃,

X is selenium, sulphur, or oxygen;

R₃ is hydroxy, —OR₂ₐ, C₁₋₈ alkyl, C₂₋₈ alkenyl, C₂₋₈ alkynyl, C₁₋₈ heteroalkyl, C₂₋₈ heteroalkenyl, C₂₋₈ heteroalkynyl, or —NR′R″;

R₂ₐ is C₁₋₈ alkyl, C₂₋₈ alkenyl, C₂₋₈ alkynyl, C₁₋₈ heteroalkyl, C₂₋₈ heteroalkenyl, C₂₋₈ heteroalkynyl, —P(═O)(OH)₂, or —P(═O)(OH)OP(═O)(OH)₂,

R′ and R″ are independently selected from the group consisting of H, C₁₋₈ alkyl, C₂₋₈ alkenyl, C₂₋₈ alkynyl, C₁₋₈ heteroalkyl, C₂₋₈ heteroalkynyl, C₂₋₈ heteroalkenyl, and C₆₋₁₀ aryl, or

NR′R″ is a substituted or unsubstituted amino acid residue;

Z comprises a heterocyclic moiety comprising at least one N, and

the symbol * indicates the point of attachment of the methylene moiety in Formula A or A′ to Z is via an available nitrogen of the heterocyclic moiety,

or a pharmaceutically acceptable salt thereof.

In some embodiments, the compound is in the form of an enantiomer, diastereomer, racemate, stereoisomer, tautomer, rotamer or a mixture thereof.

In another embodiment, B is —CH₃ or —CH₂OH.

In some embodiments, R₃ is hydroxyl.

In some embodiments, M is selected from —O—(CH₂)₂—O—C₁₋₂₄alkyl, —O—(CH₂)₃—O—C₁₋₂₄ alkyl, —O—CH₂—CH(OH)—CH₂—O—C₁₋₂₄alkyl, and —O—CH₂—CH(OH)—CH₂—S—C₁₋₂₄alkyl. In another embodiment, M is —O—(CH₂)ₐ—O—(CH₂)ₜ—CH₃, wherein a is 2 to 4 and t is 11 to 19. In some embodiments, a is 2 or 3 and t is 15 or 17. In some embodiments, M is —O—(CH₂)₂—O—(CH₂)₁₅CH₃ or —O—(CH₂)₂—O—(CH₂)₁₇CH₃. In one embodiment, M is —O—(CH₂)₃—O—(CH₂)₁₅CH₃ or —O—(CH₂)₃—O—(CH₂)₁₇CH₃.

In another embodiment, X is oxygen.

In another embodiment, R₃ is hydroxyl.

In another embodiment, Z is purine or pyrimidine.

In another embodiment, R₁′ is hydrogen and R₁ is —ORⁱ.

In another embodiment, Rⁱ is unsubstituted C₁₀₋₂₀ alkyl.

In another embodiment, Rⁱ is unsubstituted C₁₆ alkyl.

In another embodiment, Z is cytosine.

In another embodiment, m is 0, 1 or 2.

In one embodiment, R₂ and R₂′ are H.

In one embodiment, M is selected from formula a, b or c:

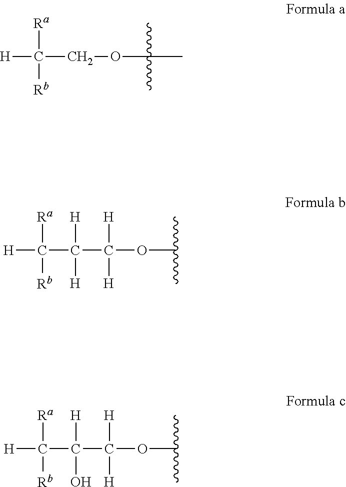

wherein R^{a} and R^{b} are independently —H, halogen, —ORⁱ, —SRⁱ, —NHRⁱ, or —NRⁱRⁱⁱ, and Rⁱ and Rⁱⁱ are independently hydrogen or an aliphatic moiety. In some embodiments, Rⁱ and Rⁱⁱ are independently —(C₁-C₂₄)alkyl, —(C₂-C₂₄)alkenyl, —(C₂-C₂₄)alkynyl or —(C₁-C₂₄)acyl.

In some embodiments, at least one of R^{a} and R^{b} is not hydrogen. In some embodiments, R^{a} and R^{b} are independently selected from the group consisting of —H, optionally substituted —O(C₁-C₂₄)alkyl, —O(C₂-C₂₄)alkenyl, —O(C₁-C₂₄)acyl, —S(C₁-C₂₄)alkyl, —S(C₂-C₂₄)alkenyl, and —S(C₁-C₂₄)acyl.

In some embodiments, for M, R₁, R₁′, R₂, R₂′, Rₓ and R_{y} are independently selected from —O(C₁-C₂₄)alkyl, —O(C₂-C₂₄)alkenyl, —O(C₂-C₂₄)alkynyl, —O(C₁-C₂₄)acyl, —S(C₁-C₂₄)alkyl, —S(C₂-C₂₄)alkenyl, —S(C₂-C₂₄)alkynyl, —S(C₁-C₂₄)acyl, —NH(C₁-C₂₄)alkyl, —NH(C₂-C₂₄)alkenyl, —NH(C₂-C₂₄)alkynyl, —NH(C₁-C₂₄)acyl, —N((C₁-C₂₄)alkyl)((C₂-C₂₄)alkyl), —N((C₁-C₂₄)alkyl)((C₂-C₂₄)alkenyl), —N((C₁-C₂₄)alkyl)((C₂-C₂₄)acyl), —N((C₁-C₂₄)alkyl)((C₂-C₂₄)alkynyl), —N((C₂-C₂₄)alkenyl)((C₂-C₂₄)alkynyl), —N((C₂-C₂₄)alkenyl)((C₂-C₂₄)alkenyl), —N((C₂-C₂₄)alkynyl)((C₂-C₂₄)alkynyl), —N((C₁-C₂₄)acyl)((C₂-C₂₄)alkynyl), or —N((C₁-C₂₄)acyl)((C₂-C₂₄)alkenyl).

In one embodiment, Z comprises (or is) purine or pyrimidine, which may be optionally substituted by at least one substituent. In some embodiments, at least one substituent may be selected from the group consisting of halogen, hydroxyl, amino, substituted amino, di-substituted amino, sulfur, nitro, cyano, acetyl, acyl, aza, C₁₋₆ alkyl, C₂₋₆ alkenyl, C₂₋₆ alkynyl, C₆₋₁₀ aryl, and carbonyl substituted with a C₁₋₆ alkyl, C₂₋₆ alkenyl, C₂₋₆ alkynyl, or C₆₋₁₀ aryl, haloalkyl and aminoalkyl.

In some embodiments, Z may be selected from adenine, 6-chloropurine, xanthine, hypoxanthine, guanine, 8-bromoguanine, 8-chloroguanine, 8-aminoguanine, 8-hydrazinoguanine, 8-hydroxyguanine, 8-methylguanine, 8-thioguanine, 2-aminopurine, 2,6-diaminopurine, thymine, cytosine, 5-fluorocytosine, uracil; 5-bromouracil, 5-iodouracil, 5-ethyluracil, 5-ethynyluracil, 5-propynyluracil, 5-propyluracil, 5-vinyluracil, or 5-bromovinyluracil. In some embodiments, Z is selected from guanine, adenine, 2,6-diaminopurine, 2-aminopurine, cytosine. In some embodiments, Z is guanine or 2,6-diaminopurine. In some embodiments, Z is cytosine and is attached to the CH₂* moiety of Formula A or A′ via the 1-position of the cytosine.

In another embodiment, Z is selected from 6-alkylpurine and N⁶-alkylpurines, N⁶-acylpurines, N⁶-benzylpurine, 6-halopurine, N⁶-acetylenic purine, N⁶-acyl purine, N⁶-hydroxyalkyl purine, 6-thioalkyl purine, N²-alkylpurines, N⁴-alkylpyrimidines, N⁴-acylpyrimidines, 4-halopyrimidines, N⁴-acetylenic pyrimidines, 4-amino and N⁴-acyl pyrimidines, 4-hydroxyalkyl pyrimidines, 4-thioalkyl pyrimidines, thymine, cytosine, 6-azapyrimidine, including 6-azacytosine, 2- and/or 4-mercaptopyrimidine, uracil, C⁵-alkylpyrimidines, C⁵-benzylpyrimidines, C⁵-halopyrimidines, C⁵-vinylpyrimidine, C⁵-acetylenic pyrimidine, C⁵-acyl pyrimidine, C⁵-hydroxyalkyl purine, C⁵-amidopyrimidine, C⁵-cyanopyrimidine, C⁵-nitropyrimidine, C⁵-aminopyrimidine, N²-alkylpurines, N²-alkyl-6-thiopurines, 5-azacytidinyl, 5-azauracilyl, triazolopyridinyl, imidazolopyridinyl, pyrrolopyrimidinyl, and pyrazolopyrimidinyl. Functional oxygen and nitrogen groups on the base can be protected as necessary or desired. Suitable protecting groups are well known to those skilled in the art, and include trimethylsilyl, dimethylhexylsilyl, t-butyldimethylsilyl, and t-butyldiphenylsilyl, trityl, alkyl groups, acyl groups such as acetyl and propionyl, methanesulfonyl, and p-toluenesulfonyl. Preferred bases include cytosine, 5-fluorocytosine, uracil, thymine, adenine, guanine, xanthine, 2,6-diaminopurine, 6-aminopurine, 6-chloropurine and 2,6-dichloropurine.

In one embodiment, Z is:

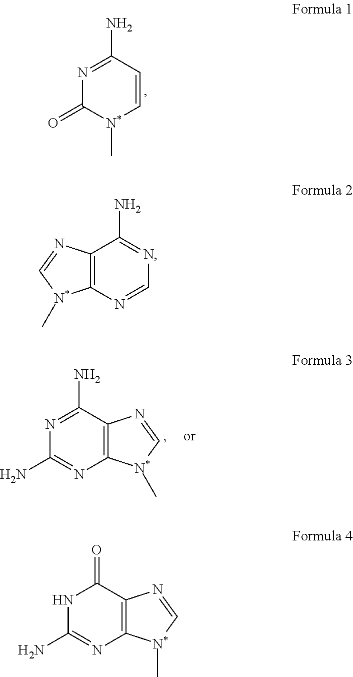

wherein the symbol * in Formulae 1-4 indicates the point of attachment of N to the methylene in Formula A or A′.

The example of Z is further described in U.S. Pat. No. 6,583,149, which is incorporated by reference in its entirety.

In one embodiment, the compound has the structure of Formula C:

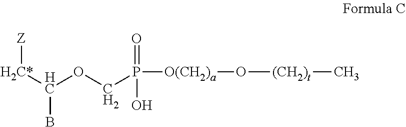

wherein:

a is 2 to 4;

t is 11 to 19; and

B is hydrogen, —CH₃, or —CH₂OH, or a pharmaceutically acceptable salt thereof.

In another embodiment, a is 2 or 3.

In another embodiment, t is 15, 16 or 17.

In another embodiment, B is —CH₂OH.

In another embodiment, Z is cytosine.

The exemplary compounds of the present invention include, but are not limited to:

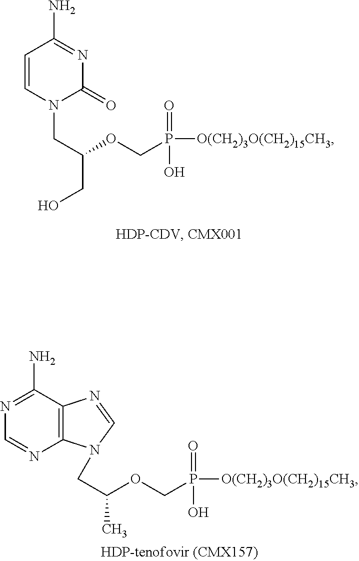

or a pharmaceutically acceptable salt thereof.

In some embodiments, R₁ is an alkoxy group having the formula —O—(CH₂)ₜ—CH₃, wherein t is 0-24. In one embodiment, t is 11-19. In another embodiment, t is 15 or 17.

Compounds, compositions, formulations, and methods of treating subjects that can be used to carry out the present invention include, but are not limited to, those described in U.S. Pat. Nos. 6,716,825, 7,034,014, 7,094,772, 7,098,197, and 7,452,898, and 7,687,480 the disclosures of which are incorporated by reference herein in their entireties.

In some embodiments, the active compounds have a phosphonate ester formed by a covalent linking of an antiviral compound selected from the group consisting of cidofovir, adefovir, cyclic cidofovir and tenofovir, to an alcohol selected from the group consisting of an alkylglycerol, alkylpropanediol, 1-S-alkylthioglycerol, alkoxyalkanol, and alkylethanediol, and pharmaceutically acceptable salts thereof.

Certain compounds of the invention possess one or more chiral centers, e.g. in the acyclic moieties, and may thus exist in optically active forms. Likewise, when the compounds contain an alkenyl group or an unsaturated alkyl or acyl moiety there exists the possibility of cis- and trans-isomeric forms of the compounds. Additional asymmetric carbon atoms can be present in a substituent group such as an alkyl group. The R- and S-isomers and mixtures thereof, including racemic mixtures as well as mixtures of cis- and trans-isomers are contemplated by this invention. All such isomers as well as mixtures thereof are intended to be included in the invention. If a particular stereoisomer is desired, it can be prepared by methods well known in the art by using stereospecific reactions with starting materials that contain the asymmetric centers and are already resolved or, alternatively, by methods that lead to mixtures of the stereoisomers and resolution by known methods.

In cases where compounds are sufficiently basic or acidic to form stable nontoxic acid or base salts, administration of the compound as a pharmaceutically acceptable salt may be appropriate. Pharmaceutically acceptable salts are salts that retain the desired biological activity of the parent compound and do not impart undesired toxicological effects. Pharmaceutically acceptable salts include those derived from pharmaceutically acceptable inorganic or organic bases and acids. Suitable salts include those derived from alkali metals such as potassium, lithium or sodium; alkaline earth metals such as calcium and magnesium; or any pharmaceutically acceptable amine salts such as a moiety containing an amino group include, for example, ammonium, mono, di, tri or tetra substituted amino groups, or any applicable organic bases containing at least one nitrogen, for example, aniline, indole, piperidine, pyridine, pyrimidine, pyrrolidine.

In some embodiments, the pharmaceutically acceptable salts are selected from organic acid addition salts formed with acids, which form a physiological acceptable anion, for example, acetate, adipate, aspartate, benzoate, besylate, bicarbonate/carbonate, bisulphate/sulphate, borate, camsylate, citrate, cyclamate, bicarbonate, carbonate, disylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinafoate salts.

Exemplary agent that may be used to form the salt include, but are not limited to, (1) acids such as inorganic acid, for example, hydrochloric acid, hydrobromic acid, sulfuric acid, phosphoric acid, nitric acid; or organic acids, for example, acetic acid, citric acid, fumaric acid, alginic acid, gluconic acid, gentisic acid, hippuric acid, benzoic acid, maleic acid, tannic acid, L-mandelic acid, orotic acid, oxalic acid, saccharin, succinic acid, L-tartaric acid, ascorbic acid, palmitic acid, polyglutamic acid, toluenesulfonic acid, naphthalenesulfonic acid, methanesulfonic acid, naphthalenedisulfonic acid, polygalacturonic acid, (2) bases such as ammonia, mono, di, tri or tetra-substituted ammonia, alkali metal bases such as potassium hydroxide, lithium hydroxide, sodium hydroxide; alkaline earth bases such as magnesium hydroxide, calcium hydroxide; organic bases such as L-arginine, diethylamine, diethylaminoethanol, dicyclohexylamine, ethylenediamine, imidazole, L-lysine, 2-hydroxyethylmorpholine, N-methyl-glucamine, potassium methanolate, zinc tert-butoxide.

One aspect of the invention provides compounds of Formula D:

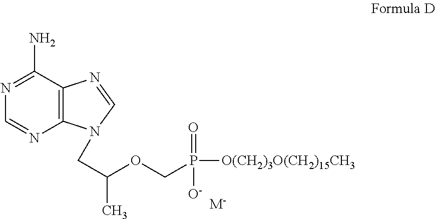

wherein M⁻ is potassium (K⁺), sodium (Na⁺), lithium (Li⁺), calcium (Ca²⁺), magnesium (Mg²⁺), or any pharmaceutically acceptable cation containing at least one nitrogen. Exemplary cations containing at least one nitrogen include, but are not limited to, various ammonium, mono, di, tri or tetra substituted amino cations. In one embodiment, the cations containing at least one nitrogen may be represented by the formula of [NR₁R₂R₃R₄]⁺ and R₁, R₂, R₃, and R₄ are independently hydrogen or aliphatic moiety. In one embodiment, the aliphatic moiety is selected from C₁₋₅ alkyl (e.g., NH₄⁺, NH₃CH₃⁺, NH₃CH₂CH₃⁺, etc), C₂₋₅ alkenyl, or C₂₋₅ alkynyl, etc. In another embodiment, the compound of Formula D is a salt selected from the group consisting of: methylamine, ethylamine, ethanolamine, tris(hydroxymethyl)aminomethane, ethylenediamine, dimethylamine, diethylamine, diisopropylamine, dibutylamine, di-sec-butylamine, dicyclohexylamine, diethanolamine, meglumine, pyrrolidine, piperidine, piperazine, benzathine, trimethylamine, triethylamine, triethanolamine, 1-(2-hydroxyethyl)-pyrrolidine, choline, tetra-methylammonium, and tetraethylammonium.

In some embodiments, M⁺ is potassium (K⁺), sodium (Na⁺), or lithium (Li⁺). In one embodiment, M⁺ is K⁺. For compounds of formula I, when M⁺ is a cation with multiple charges, multiple equivalents of anions will present to meet the cation-anion balance. For example, when the cation is Ca²⁺ or Mg²⁺, two equivalents of the anions are present to meet the requirement for cation-anion balance.

In one embodiment, the compound has the structure of:

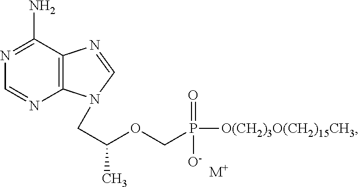

The salt may be in various forms, all of which are included within the scope of the invention. These forms include anhydrous form or solvates. In one embodiment, M⁺ is K⁺, Na⁺, or Li⁺. In other embodiments, the salt may be in the crystalline form with various degrees. In one embodiment, the compound is in an anhydrous form, a solvate or crystalline form.

Active compounds as described herein can be prepared in accordance with known procedures, or variations thereof that will be apparent to those skilled in the art. See, e.g., Painter et al., Evaluation of Hexadecyloxypropyl-9-R-[2-(Phosphonomethoxy)Propyl]-Adenine, CMX157, as a Potential Treatment for Human Immunodeficiency Virus Type 1 and Hepatitis B Virus Infections, Antimicrobial Agents and Chemotherapy 51, 3505-3509 (2007) and US Patent Application Publication No. 2007/0003516 to Almond et al.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The process to be utilized in the preparation of the compounds described herein depends upon the specific compound desired. Such factors as the selection of the specific substituent and various possible locations of the specific substituent all play a role in the path to be followed in the preparation of the specific compounds of this invention. Those factors are readily recognized by one of ordinary skill in the art.

In general, the compounds of this invention may be prepared by standard techniques known in the art and by known processes analogous thereto. General methods for preparing compounds of the present invention are set forth below.

In the following description, all variables are, unless otherwise noted, as defined in the formulas described herein. The following non-limiting descriptions illustrate the general methodologies that may be used to obtain the compounds described herein.

Compounds (or “prodrugs”) useful in the invention can be prepared in a variety of ways, as generally depicted in Schemes I-VI and examples of U.S. Pat. No. 6,716,825. The general phosphonate esterification methods described below are provided for illustrative purposes only and are not to be construed as limiting this invention in any manner. Indeed, several methods have been developed for direct condensation of phosphonic acids with alcohols (see, for example, R. C. Larock, Comprehensive Organic Transformations, VCH, New York, 1989, p. 966 and references cited therein). Isolation and purification of the compounds and intermediates described in the examples can be effected, if desired, by any suitable separation or purification procedure such as, for example, filtration, extraction, crystallization, flash column chromatography, thin-layer chromatography, distillation or a combination of these procedures. Specific illustrations of suitable separation and isolation procedures are in the examples below. Other equivalent separation and isolation procedures can of course, also be used.

Scheme I of U.S. Pat. No. 6,716,825 outlines a synthesis of bisphosphonate prodrugs that contain a primary amino group, such as pamidronate or alendronate. Example 1 therein provides conditions for a synthesis of 1-O-hexadecyloxypropyl-alendronate (HDP-alendronate) or 1-O-hexadecyloxypropyl-pamidronate (HDP-pamidronate). In this process, a mixture of dimethyl 4-phthalimidobutanoyl phosphonate (1b, prepared as described in U.S. Pat. No. 5,039,819)) and hexadecyloxypropyl methyl phosphite (2) in pyridine solution is treated with triethylamine to yield bisphosphonate tetraester 3b which is purified by silica gel chromatography. Intermediate 2 is obtained by transesterification of diphenyl phosphite as described in Kers, A., Kers, I., Stawinski, J., Sobkowski, M., Kraszewski, A. Synthesis, April 1995, 427 430. Thus, diphenyl phosphite in pyridine solution is first treated with hexadecyloxypropan-1-ol, then with methanol to provide compound 2.

An important aspect of the process is that other long chain alcohols may be used in place of hexadecyloxypropan-1-ol to generate the various compounds of this invention. Treatment of intermediate 3b with bromotrimethylsilane in acetonitrile cleaves the methyl esters selectively to yield monoester 4b. Treatment of 4b with hydrazine in a mixed solvent system (20% methanol/80% 1,4-dioxane) results in removal of the phthalimido protecting group as shown. The desired alendronate prodrug is collected by filtration and converted to the triammonium salt by treatment with methanolic ammonia.

Scheme II of U.S. Pat. No. 6,716,825 illustrates a synthesis of analogs of bisphosphonates lacking a primary amino group, in this case the process steps are similar to those of Scheme 1 except that protection with a phthalimido group and subsequent deprotection by hydrazinolysis are unnecessary. Bisphosphonates having 1-amino groups, such as amino-olpadronate, may be converted to analogs according to the invention prodrugs using a slightly modified process shown in Scheme III of U.S. Pat. No. 6,716,825. Treatment of a mixture of compound 2 and 3-(dimethylamino)propionitrile with dry HCl followed by addition of dimethyl phosphite affords tetraester 3 which, after demethylation with bromotrimethylsilane, yields hexadecyloxypropyl-amino-olpadronate.

Scheme IV of U.S. Pat. No. 6,716,825 illustrates synthesis of a bisphosphonate analog where the lipid group is attached to a primary amino group of the parent compound rather than as a phosphonate ester.

Scheme V of U.S. Pat. No. 6,716,825 illustrates a general synthesis of alkylglycerol or alkylpropanediol analogs of cidofovir, cyclic cidofovir, and other phosphonates. Treatment of 2,3-isopropylidene glycerol, 1, with NaH in dimethylformamide followed by reaction with an alkyl methanesulfonate yields the alkyl ether, 2. Removal of the isopropylidene group by treatment with acetic acid followed by reaction with trityl chloride in pyridine yields the intermediate 3. Alkylation of intermediate 3 with an alkyl halide results in compound 4. Removal of the trityl group with 80% aqueous acetic acid affords the O,O-dialkyl glycerol, 5. Bromination of compound 5 followed by reaction with the sodium salt of cyclic cidofovir or other phosphonate-containing nucleotide yields the desired phosphonate adduct, 7. Ring-opening of the cyclic adduct is accomplished by reaction with aqueous sodium hydroxide. The compound of propanediol species may be synthesized by substituting 1-O-alkylpropane-3-ol for compound 5 in Scheme V. The tenofovir and adefovir analogs may be synthesized by substituting these nucleotide phosphonates for cCDV in reaction (f) of Scheme V. Similarly, other nucleotide phosphonates of the invention may be formed in this manner.

Scheme VI of U.S. Pat. No. 6,716,825 illustrates a general method for the synthesis of nucleotide phosphonates of the invention using 1-O-hexadecyloxypropyl-adefovir as the example. The nucleotide phosphonate (5 mmol) is suspended in dry pyridine and an alkoxyalkanol or alkylglycerol derivative (6 mmol) and 1,3-dicyclohexylcarbodiimde (DCC, 10 mmol) are added. The mixture is heated to reflux and stirred vigorously until the condensation reaction is complete as monitored by thin-layer chromatography. The mixture is then cooled and filtered. The filtrate is concentrated under reduced pressure and the residues adsorbed on silica gel and purified by flash column chromatography (elution with approx. 9:1 dichloromethane/methanol) to yield the corresponding phosphonate monoester.

Scheme VII (which is referenced as FIG. 1 in Kern et al., AAC 46 (4):991) illustrates the synthesis for alkoxyalkyl analogs of cidofovir (CDV) and cyclic cidofovir (cCDV). In FIG. 1, the arrows indicate the following reagents: (a) N,N-dicyclohexylmorpholinocarboxamide, N,N-dicyclohexylcarbodiimide, pyridine, 100° C.; (b) 1-bromo-3-octadecyloxyethane (ODE), or 1-bromo-3-hexadecyloxypropane (HDP), N,N-dimethylformamide, 80° C.; (c) 0.5 M NaOH.

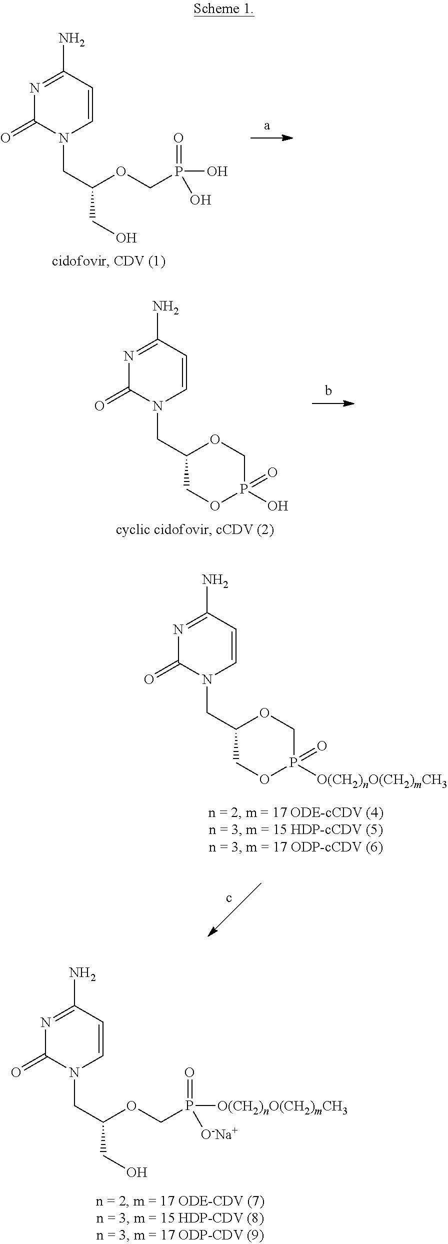

As described herein, compounds of the invention may optionally be substituted with one or more substituents, such as are illustrated generally above, or as exemplified by particular classes, subclasses, and species of the invention. In general, the term “substituted” refers to the replacement of hydrogen radicals in a given structure with the radical of a specified substituent. Unless otherwise indicated, a substituted group may have a substituent at each substitutable position of the group, and when more than one position in any given structure may be substituted with more than one substituent selected from a specified group, the substituent may be either the same or different at every position. Combinations of substituents envisioned by this invention may be those that result in the formation of stable or chemically feasible compounds.

In cases where compounds are sufficiently basic or acidic to form stable nontoxic acid or base salts, administration of the compound as a pharmaceutically acceptable salt may be appropriate. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. In particular, examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

In one embodiment, the present invention is a pharmaceutical composition comprising the compounds described herein. In another embodiment, the pharmaceutical composition further comprises a pharmaceutically acceptable carrier. The term “pharmaceutically acceptable carrier” as used herein refers to any substance, not itself a therapeutic agent, used as a vehicle for delivery of a therapeutic agent to a subject. Examples of pharmaceutically acceptable carriers and methods of manufacture for various compositions include, but are not limited to, those described in Remington's Pharmaceutical Sciences, 18th Ed., Mack Publishing Co. (1990) (See also U.S. Patent Application US 2007/0072831).

In some embodiments, the pharmaceutical composition further comprises one or more immunosuppressive agents.

While it is possible for the active ingredients to be administered alone it is preferably to present them as pharmaceutical formulations. The formulations, both for veterinary and for human use, of the present invention comprise at least one active ingredient, as above defined, together with one or more pharmaceutically acceptable carriers (excipients, diluents, etc.) thereof and optionally other therapeutic ingredients. The carrier(s) must be “acceptable” in the sense of being compatible with the other ingredients of the formulation and not deleterious to the recipient thereof.

The compounds of the invention may be formulated with conventional carriers, diluents and excipients, which will be selected in accord with ordinary practice. Tablets will contain excipients, glidants, fillers, binders, diluents and the like. Aqueous formulations are prepared in sterile form, and when intended for delivery by other than oral administration generally will be isotonic. Formulations optionally contain excipients such as those set forth in the “Handbook of Pharmaceutical Excipients” (1986) and include ascorbic acid and other antioxidants, chelating agents such as EDTA, carbohydrates such as dextrin, hydroxyalkylcellulose, hydroxyalkylmethylcellulose, stearic acid and the like.

Any suitable route of administration may be employed for providing a mammal, especially a human with an effective dosage of a compound of the present invention. For example, the compositions of the present invention may be suitable for formulation for oral, parenteral (including subcutaneous, intramuscular, intravenous, intradermal, intrathecal and epidural), inhalation spray, topical, rectal, nasal, sublingual, buccal, vaginal or implanted reservoir administration, etc. In some embodiments, the compositions are administered orally, topically, intraperitoneally or intravenously. These suspensions may be formulated according to techniques known in the art using suitable dispersing or wetting agents and suspending agents. Among the acceptable vehicles and solvents that may be employed are water, Ringer's solution and isotonic sodium chloride solution. In addition, sterile, fixed oils are conventionally employed as a solvent or suspending medium.

Compounds of the invention and their physiologically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient.

A pharmaceutically acceptable oil may be employed as a solvent or suspending medium in compositions of the present invention. Fatty acids, such as oleic acid and its glyceride derivatives are suitably included in injectable formulations, as are natural pharmaceutically acceptable oils, such as olive oil or castor oil, especially in their polyoxyethylated versions. The oil containing compositions of the present invention may also contain a long-chain alcohol diluent or dispersant, such as carboxymethyl cellulose or similar dispersing agents that are commonly used in the formulation of pharmaceutically acceptable dosage forms including emulsions and suspensions. The compositions suitably further comprise surfactants (such as non-ionic detergents including Tween® or Span®) other emulsifying agents, or bioavailability enhancers.

The compositions of this invention may be in the form of an orally acceptable dosage form including, but not limited to, capsules, tablets, suspensions or solutions. The oral dosage form may include at least one excipient. Excipients used in oral formulations of the present can include diluents, substances added to mask or counteract a disagreeable taste or odor, flavors, dyes, fragrances, and substances added to improve the appearance of the composition. Some oral dosage forms of the present invention suitably include excipients, such as disintegrants, binding agents, adhesives, wetting agents, polymers, lubricants, or glidants that permit or facilitate formation of a dose unit of the composition into a discrete article such as a capsule or tablet suitable for oral administration. Excipient-containing tablet compositions of the invention can be prepared by any suitable method of pharmacy which includes the step of bringing into association one or more excipients with a compound of the present invention in a combination of dissolved, suspended, nanoparticulate, microparticulate or controlled-release, slow-release, programmed-release, timed-release, pulse-release, sustained-release or extended-release forms thereof.

Formulations of the present invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents such as pentamidine for treatment of pneumocystis pneumonia.

Formulations suitable for vaginal administration may be presented as pessaries, rings, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described. Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

Pharmaceutically acceptable compositions of the present invention may be in the form of a topical solution, ointment, or cream in which the active component is suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Where the topical formulation is in the form of an ointment or cream, suitable carriers include, but are not limited to, mineral oil, sorbitan monostearate, polysorbate 60, cetyl esters wax, cetearyl alcohol, 2 octyldodecanol, benzyl alcohol and water. In some embodiments, the topical composition of the present invention is in the form of a spray.

The pharmaceutically acceptable compositions of this invention may also be administered by nasal, aerosol or by inhalation administration routes. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. In some embodiments, the nasal administration of the composition of the present invention is in the form of a spray. Any suitable carrier for spray application may be used in the present invention.

Alternatively, pharmaceutically acceptable compositions of this invention may be in the form of a suppository for rectal administration. The suppositories can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Additionally, the pharmaceutical formulation including compounds of the present invention can be in the form of a parenteral formulation. The term “parenteral” as used herein includes subcutaneous, intravenous, intramuscular, intra-articular, intra-synovial, intrasternal, intrathecal, intrahepatic, intralesional and intracranial injection or infusion techniques.

In certain embodiments, the pharmaceutically compositions of this invention are formulated for oral administration. For oral administration to humans, the dosage range is 0.01 to 1000 mg/kg body weight in divided doses. In one embodiment the dosage range is 0.1 to 100 mg/kg body weight in divided doses. In another embodiment the dosage range is 0.5 to 20 mg/kg body weight in divided doses. For oral administration, the compositions may be provided in the form of tablets or capsules containing 1.0 to 1000 milligrams of the active ingredient, particularly, 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated.

It should also be understood that a specific dosage and treatment regimen for any particular patient will depend upon a variety of factors, including the activity of the specific compound employed, the mode of administration, the age, body weight, general health, gender, diet, rate of excretion, drug combination, and the judgment of the treating physician, the condition being treated and the severity of the condition. Such dosage may be ascertained readily by a person skilled in the art. This dosage regimen may be adjusted to provide the optimal therapeutic response.

The present invention further provides veterinary compositions comprising at least one active ingredient as above defined together with a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention can be used to provide controlled release pharmaceutical formulations containing as active ingredient one or more compounds of the invention (“controlled release formulations”) in which the release of the active ingredient can be controlled and regulated to allow less frequent dosing or to improve the pharmacokinetic or toxicity profile of a given invention compound. Controlled release formulations adapted for oral administration in which discrete units comprising one or more compounds of the invention can be prepared according to conventional methods. Controlled release formulations may be employed for treating various viral infections and/or diseases associated with virus. An exemplary viral infection is an orthopox infection, such as smallpox.

Pharmacokinetic enhancers. The compounds of the invention may be employed in combination with pharmacokinetic enhancers (sometimes also referred to as “booster agents”). One aspect of the invention provides the use of an effective amount of an enhancer to enhance or “boost” the pharmacokinetics of a compound of the invention. An effective amount of an enhancer, for example, the amount required to enhance an active compound or additional active compound of the invention, is the amount necessary to improve the pharmacokinetic profile or activity of the compound when compared to its profile when used alone. The compound possesses a better efficacious pharmacokinetic profile than it would without the addition of the enhancer. The amount of pharmacokinetic enhancer used to enhance the potency of the compound is, preferably, subtherapeutic (e.g., dosages below the amount of booster agent conventionally used for therapeutically treating infection in a patient). An enhancing dose for the compounds of the invention is subtherapeutic for treating infection, yet high enough to effect modulation of the metabolism of the compounds of the invention, such that their exposure in a patient is boosted by increased bioavailability, increased blood levels, increased half life, increased time to peak plasma concentration, increased/faster inhibition of HIV integrase, RT or protease and/or reduced systematic clearance. One example of a pharmacokinetic enhancer is RITONAVIR™ (Abbott Laboratories).

Combinations. The compounds and methods of the present invention can be used to treat or to prevent an orthopox virus infection (e.g., smallpox) in a subject where vaccination is not appropriate. For example, the present methods are provided for subjects who are immunocompromised, for example, due to HIV infection, immunosuppressive drug therapy for organ transplantation, or chemotherapy for cancer treatment. As noted above, the compositions of the present invention can include the active compounds as described above in combination with one or more (e.g., 1, 2, 3) immunosuppressant agents such as described below, in analogous manner as known in the art.

Specific examples of such combinations include, but are not limited to: CMX001 or a pharmaceutically acceptable salt thereof in combination with at least one immunosuppressant agent. Exemplary immunosuppressant agents include, but are not limited to, Daclizumab, Basiliximab, Tacrolimus, Sirolimus, Mycophenolate (as sodium or mofetil), Cyclosporine A, Glucocorticoids, Anti-CD3 monoclonal antibodies (OKT3), Antithymocyte globulin (ATG), Anti-CD52 monoclonal antibodies (campath 1-H), Azathioprine, Everolimus, Dactinomycin, Cyclophosphamide, Platinum, Nitrosurea, Methotrexate, Azathioprine, Mercaptopurine, Muromonab, IFN gamma, Infliximab, Etanercept, Adalimumab, Tysabri (Natalizumab), Fingolimodm or a combination thereof. In some embodiments, the pharmaceutical composition includes CMX001, Tysabri (natalizumab), and a pharmaceutically acceptable carrier.

In one embodiment, the pharmaceutical composition described herein comprises CMX001, or pharmaceutically acceptable salt thereof and one or more medication that causes PML in at least one pharmaceutically acceptable carrier. In one embodiment, one or more medication is selected from the group consisting of Rituxan, Raptiva, Tysabri (natalizumab), Myfortic, Avonex, Remicade, Enbrel, Humira, Cellcept and a combination thereof in at least one pharmaceutically acceptable carrier.

In another embodiment, the pharmaceutical composition described herein includes CMX001 or a pharmaceutically acceptable salt of any thereof, in at least one pharmaceutically acceptable carrier.

One aspect of the present invention provides methods of treating conditions/disease associated with at least one virus in a subject which includes administering to the subject a therapeutically effective amount of a compound described herein.

In one embodiment, the compounds described herein specifically target against viral replication and/or virally infected/transformed cells. In one embodiment, the compounds described herein have a higher cytotoxicity against virally infected and/or transformed cells compared to normal (uninfected cells).

In another embodiment, the disease is associated with at least one virus selected from the group consisting of variola major and minor, vaccinia, smallpox, cowpox, camelpox, monkeypox, molluscum contagiosum and combinations thereof.

In one embodiment, the subject is human. In one embodiment, the subject is an immunocompromised subject. In one embodiment, the subject is in need of a chemotherapy agent.

In some embodiments, the subject has been previously treated with at least one antiviral agent and the previous treatment has failed and the previously used antiviral agent is cidofovir. In another embodiment, the present invention provides methods of treating conditions/disease associated with at least one virus in a subject, wherein treatment with cidofovir alone has failed.

In another embodiment, the disease is associated with at least one orthopox virus and the methods comprise administering a compound (CMX001) having the structure:

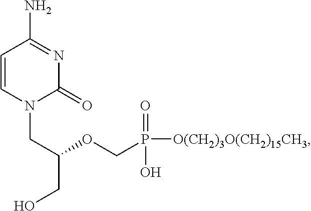

or a pharmaceutically acceptable salt thereof.

As used herein, immunodeficiency (or immune deficiency) is a state in which the immune system's ability to fight infectious disease is compromised or entirely absent. An immunocompromised subject is a subject that has an immunodeficiency of any kind or of any level. An immunocompromised person may be particularly vulnerable to opportunistic infections, in addition to normal infections. Exemplary immunocompromised subject includes, but are not limited to, a subject with primary immunodeficiency (a subject that is born with defects in immune system) and a subject with secondary (acquired) immunodeficiency. In addition, other common causes for secondary immunodeficiency include, but are not limited to, malnutrition, aging and particular medications (e.g. immunosuppressive therapy, such as chemotherapy, disease-modifying antirheumatic drugs, immunosuppressive drugs after organ transplants, glucocorticoids). Other exemplary diseases that directly or indirectly impair the immune system include, but are not limited to, various types of cancer, (e.g. bone marrow and blood cells (leukemia, lymphoma, multiple myeloma)), acquired immunodeficiency syndrome (AIDS) caused by human immunodeficiency virus (HIV), chronic infections and autoimmune diseases (e.g. Acute disseminated encephalomyelitis (ADEM), Addison's disease, Alopecia areata, Ankylosing spondylitis, Antiphospholipid antibody syndrome (APS), Autoimmune hemolytic anemia, Autoimmune hepatitis, Autoimmune inner ear disease, Bullous pemphigoid, Coeliac disease, Chagas disease, Chronic obstructive pulmonary disease, Crohns Disease, Dermatomyositis, Diabetes mellitus type 1, Endometriosis, Goodpasture's syndrome, Graves' disease, Guillain-Barré syndrome (GBS), Hashimoto's disease, Hidradenitis suppurativa, Kawasaki disease, IgA nephropathy, Idiopathic thrombocytopenic purpura, Interstitial cystitis, Lupus erythematosus, Mixed Connective Tissue Disease, Morphea, Multiple sclerosis (MS), Myasthenia gravis, Narcolepsy, Neuromyotonia, Pemphigus vulgaris, Pernicious anaemia, Psoriasis, Psoriatic Arthritis, Polymyositis, Primary biliary cirrhosis, Rheumatoid arthritis, Schizophrenia, Scleroderma, Sjögren's syndrome, Stiff person syndrome, Temporal arteritis (also known as “giant cell arteritis”), Ulcerative Colitis, Vasculitis, Vitiligo, Wegener's granulomatosis.)

In some embodiments, the compound described herein is administered to said subject at a dosage of less than 1 mg/kg; in some embodiments the conjugate compound is administered to said subject at a dosage of 0.01, 0.05, 0.1, 0.2, 0.3, or 0.5 to 5, 10, 15 or 20 mg/kg.

In one embodiment, the subject is a transplant patient (including, but is not limited to, a renal transplant patient, a bone marrow transplant patient, a hepatic transplant patient, a liver transplant patient, a stem cell transplant patient, a lung transplant patient, a pancreas transplant patient, and/or a heart transplant patient) on immunosuppressive agent.

In some embodiments, the present invention is applied to a subject on immunosuppressive medications, (e.g. transplant patient or subjects that are suffering from an over-active immune system), a subject receiving certain kinds of chemotherapy, or a subject that is infected with human immunodeficiency virus (HIV). In one embodiment, the present invention is applied to a subject on at least one chemotherapy medication.

Additional exemplary immunosuppressant agents are further described in Mukherjee et al., A comprehensive review of immunosuppression used for liver transplantation, Journal of Transplantation, vol. 2009, article ID 701464 and Woodroffe et al., Clinical and cost-effectiveness of newer immunosuppressive regimens in renal transplantation: a systematic review and modeling study, Health Technology Assessment, vol. 9, No. 21(2005).

The present invention will now be described in more detail with reference to the following examples. However, these examples are given for the purpose of illustration and are not to be construed as limiting the scope of the invention.

### EXAMPLES

### Example 1

The antiviral activity of CMX001 has been characterized against orthopoxviruses in vitro and in vivo in mice, rabbits, and non-human primates. The in vitro potency of CMX001 against variola virus is 0.1 μM and ranges from 0.5 to 0.9 μM against cowpox, vaccinia, ectromelia, and rabbitpox viruses (Hostetler, 2009). In mice, CMX001 is effective in preventing mortality after intranasal infection with a lethal inoculum of ectromelia, cowpox, vaccinia, or monkeypox virus when administered several days after infection. Effective doses are in the range of 1 mg/kg to 20 mg/kg once per day for 5 days. Alternatively, a single dose of 20 mg/kg to 100 mg/kg is effective in some cases. In the rabbit model, CMX001 is also effective in preventing mortality after a lethal infection with rabbitpox virus. Effective doses ranged from 1 mg/kg twice daily for 5 days to 20 mg/kg once daily for 5 days. A single dose of 20 mg/kg dose is also effective in some cases. In a recent randomized, blinded, placebo-controlled study of rabbitpox-infected rabbits, where treatment was initiated after the onset of lesions, three doses of 20 mg/kg administered every other day (60 mg/kg total dose) provided statistically significant protection from mortality after intradermal inoculation of rabbits with a lethal dose of rabbitpox virus (11/12 survivors in the CMX001 group versus 2/12 in the placebo group).

Because of differences in metabolism and exposure to CMX001 in non-human primates, studies of CMX001 in cynomolgus monkeys are not relevant to humans. However, cidofovir has been shown to be efficacious in monkey models of infection and both cidofovir and CMX001 deliver the identical active antiviral species, cidofovir diphosphate. For example, monkeys were protected from mortality after a lethal intravenous inoculation of monkeypox virus when cidofovir was administered at 20 mg/kg on days 1, 6, and 11 after infection; there were 7/8 survivors in the cidofovir group versus 1/8 survivors in the placebo group (Huggins, 2004).

### Example 2

Progressive vaccinia (PV), previously known as vaccinia necrosum, vaccinia gangrenosum, or disseminated vaccinia, is a rare, often fatal adverse event after vaccination with smallpox vaccine, made from live vaccinia virus. During recent vaccination programs potential cases of PV were investigated, but none met standard case definitions. PV has not been confirmed to have occurred in the United States since 1987. On Mar. 2, 2009, a U.S. Navy Hospital contacted the Poxvirus Program at CDC to report a possible case of PV in a male military smallpox vaccinee. The service member had been newly diagnosed with acute mylegenous leukemia M0 (AML M0). During evaluation for a chemotherapy-induced neutropenic fever, he was found to have an expanding and nonhealing painless vaccination site 6.5 weeks after receipt of smallpox vaccine. Clinical and laboratory investigation confirmed that the vaccinee met the Brighton Collaboration and CDC adverse event surveillance guideline case definition for PV. The following summarizes the patient's protracted clinical course. The quantities of investigational and licensed therapeutics and diagnostics used were greater than anticipated based on existing smallpox preparedness plans.

On Jan. 13, 2009, a healthy service member aged 20 years received a primary smallpox vaccination (ACAM2000 [Acambis, Inc., Cambridge, Mass.]) in accordance with the U.S. Department of Defense smallpox vaccination policy; no other vaccinations were administered that day. Twelve days later, the patient visited a local hospital with fever and headache of 1 day's duration and was admitted for workup of leukopenia after his white blood cell count was found to be 1,400 cells/mm³. On Jan. 28, after transfer to a U.S. Navy tertiary-care facility, he was diagnosed with AML M0. On January 30 and February 13, the patient underwent two successive rounds of induction chemotherapy with cytarabine, idarubicin, and dexamethasone. Before initial chemotherapy, the vaccination site pustule had a central crust and measured approximately 1 cm in diameter with minimal surrounding erythema. During the patient's hospital stay from the end of January to the beginning of March, his vaccination site dressing was changed daily.

On March 2, during the evaluation of neutropenic fever, the failure of the patient's vaccination site to heal was described. An annular lesion with a deep bulla, raised violaceous leading edge, and a central crust that bled with pressure was noted. The size of the lesion had progressed to approximately 4×4 cm with minimal surrounding erythema or induration. The patient described no pain at the site, although he reported occasional pruritus. A swab of the lesion and serum were sent to CDC for viral and serologic analysis. Viral analysis of the swab by multiple real-time polymerase chain reaction (PCR) assays for orthopoxvirus and vaccinia yielded evidence of viral DNA; viral culture was positive for orthopoxvirus. Serum showed equivocal to absent levels of anti-orthopoxvirus immunoglobulin G (IgG) and immunoglobulin M (IgM) by enzyme-linked immunosorbent assay. The results of the diagnostic testing combined with the patient's medical history met the PV level 1 case definition as defined by the Brighton Collaboration and the confirmed case definition as described by CDC surveillance guidelines. The criteria met by both case definitions were 1) a documented clinical diagnosis of a disease that is known to be associated with cell-mediated immunodeficiency (in this case AML M0), 2) the primary vaccination site's failure to resolve (in this case >6 weeks post vaccination), and 3) the laboratory confirmation of vaccinia virus as the causative agent.

On March 3, imiquimod was applied directly to the lesion. Within 24 hours of confirmation of PV on March 4, the patient received licensed Vaccinia Immune Globulin Intravenous (Human) (VIGIV) (Cangene Corporation, Winnipeg, Canada). On March 5 and March 6, oral and topical ST-246 (SIGA Technologies, Corvallis, Oreg.) were administered under an Emergency Investigational New Drug (E-IND) application. The patient remained stable until the evening of March 7, when he became septic with Pseudomonas aeruginosa, likely from a perirectal abscess. He required intubation, maximal vasopressor support, multiple antibiotics, and stress dose corticosteroids. He then developed multiorgan failure and began continuous venovenous hemodialysis. During the next 12 days, the patient slowly stabilized. As a consequence of the duration and amount of vasopressor support, the patient required a bilateral trans-tibial amputation because of dry gangrene of his feet.

During March 6-19, the patient received additional oral and topical ST-246 and VIGIV; his ST-246 levels were noted to be lower than those achieved both in healthy subjects in phase I clinical trials and in successful treatment of nonhuman primates with systemic orthopoxvirus disease. The lesion size remained unchanged, but the central crust of the vaccination site sloughed off, followed by most of the outer “ring” flattening, leaving a shallow ulcer with healthy-appearing granulation tissue. During his steroid taper, additional satellite lesions surrounding the vaccination site appeared on March 18, and viral DNA was detected again in the blood. These lesions became vesicular in nature, and on March 26, after a second E-IND was issued, CMX001 (Chimerix, Inc., Research Triangle Park, North Carolina), a lipid conjugate of cidofovir, was administered.

From March 24 onward, the satellite and main vaccination site lesions continued to crust, the scabs separated, and underlying tissue epithelialized. Blood viral DNA levels cleared on March 29. On April 10, the borders of lesions again appeared raised; a shave biopsy grew methicillin-resistant Staphylococcus aureus, which responded to antibiotic therapy. The patient received intermittent granulocyte colony-stimulating factor, and his absolute neutrophil and lymphocyte count increased over time. By May 1, significant portions of the scabs/eschars had fallen off or were removed manually, revealing healthy epidermis. Numerous therapeutics with different biologic mechanisms were used to treat PV in this patient (Table 1).

From February 21 onward, the patient had remained in contact isolation, first for a Clostridium difficile infection and then for his progressive vaccinia infection. On May 5, contact precautions were discontinued because of the lack of viable virus in lesion specimens from the previous 4 weeks. No cases of contact vaccinia were identified among this patient's health-care workers or close contacts.

During March 3-May 18, nearly 200 clinical specimens (lesion and satellite swabs/crusts, ethylenediaminetetraacetic acid [EDTA] blood, bone marrow, and serum) were collected and submitted to CDC to evaluate disease progression and guide therapeutic interventions. After April 23, swabs from satellite lesions or the main vaccination site showed significantly reduced or absent levels of viral DNA, and no viable virus was detected after April 2. Oropharyngeal sampling and bone marrow biopsies from early and late March, respectively, were negative for vaccinia virus. Orthopoxvirus DNA was detected in EDTA blood at intermittent times during the course of the patient's infection; however, no viable virus was cultured from blood. As of May 12, the patient had no demonstrable IgM response to orthopoxvirus; IgG levels appeared fully reliant on VIGIV infusion.

Although PV is a rare adverse event (one case per million during routine vaccination during 1963-1968), its case fatality rate in primary U.S. vaccinees was 15% despite treatment with massive amounts of VIG (intramuscular). Extensive surgical debridement was sometimes required, even necessitating disarticulation of the arm to “debulk” the amount of infectious material. Before smallpox vaccination, patients are screened for numerous contraindications. At the time of his vaccination, the patient described in this report did not have any obvious signs or symptoms that would meet any exclusion criteria for vaccination. Training in use of, and careful adherence to, screening tools can identify vaccine candidates at risk for PV and other adverse events. Despite this, vaccinees with occult immunodeficiencies might not be recognized, and therefore appropriately deferring vaccination in these persons is not always possible.

Lack of inflammation at the expanding vaccination site is the hallmark of PV. Any smallpox vaccinee who has an expanding, nonhealing, painless vaccination site without inflammation for more than 2 weeks should be evaluated for an underlying immunodeficiency, and diagnosis of and treatment for PV should be considered.

This patient's protracted clinical course is consistent with previously published cases reports and surveillance summaries. The development of progressive vaccinia, historically observed in patients with cellular immunodeficiencies, often leads to superinfection and subsequent sepsis (i.e., fungal, parasitic, and bacterial infections resulting in toxic or septicemic shock, then ultimately death). Past treatment typically included massive doses of VIG, administration of thiosemicarbazone, blood products, and supportive care for accompanying infections (7,9). The improvement of progressive vaccinia in this patient was associated with receipt of VIGIV (the only licensed product for treatment of vaccinia adverse events stockpiled by the SNS), ST-246, and CMX001, and an increase in lymphocyte count. The use of two antiviral agents with different mechanisms of action was enabled by the research and development of medical countermeasures for smallpox preparedness activities, as well as the use of the emergency IND process. As of May 18, the patient had shed nearly all of the scab material on and around the vaccination site.

The patient received VIGIV in the amount originally estimated to treat 30 persons. The extraordinary amounts of VIGIV used to treat this single case of PV underscore the need to reevaluate the adequacy of the national stockpiled supply of this or other medical countermeasures (treatment or prophylactic).

TABLE 1
Administration, dates and dosages of therapeutics used in treatment
of progressive vaccinia in a military smallpox vaccine - United States,
Mar. 5-May 18, 2009

| | | | | Administration |
|---|---|---|---|---|
| Treatment* | Formulation | Dosage | Application | Dates |
| ST-246 | Oral | 400 mg | Once daily | March 5-19 |
| | | 800 mg | Once daily | March 20-24 |
| | | 1200 mg | Once daily | March 25 to |
| | | | | present^{a} |
| ST-246 | Topical | 1%, 0.5 mL | Once daily | March 6; April |
| | | | | 21-May 12 |
| | | 1%, 0.5 mL | Twice daily | March 7-April |
| | | | | 20 |
| CMX001 | Oral | 200 mg | Once per | March 26 |
| | | | date | |
| | | 100 mg | Once per | April 1, 7, 13, |
| | | | date | 20, 27 |
| Imiquimod | Topical | 5%, 12.5 mg | Once daily | March 24-May |
| | | | | 12 |
| VIGIV^{b,c,d} | Intravenous | 6,000 U/kg | Once per | March 4, 11, |
| | | | date | 20; April 1, |
| | | | | 3, 6, 8, 18 |
| | | 18,000 U/kg | Once per | April 9 |
| | | | date | |
| | | 24,000 U/kg | Once per | March 24; |
| | | | date | April 14, 23, |
| | | | | 28; May 8 |

^{a}as of May 18, 2009.
^{b}Vaccinia Immune Globulin Intravenous (Human).
^{c}VIGIV is supplied as a 15 mL single dose vial containing >50,000 U/vial.
^{d}Patient received a total of 16,740,000 U of VIGIV during March 4-May 8.

In summary, the patient's dose of oral ST-246 was increased twice to obtain more optimal drug levels, CMX001 was begun, topical ST-246 and imiquimod continued, as well as periodic infusions of VIGIV at varying doses.

### Example 3

A study was completed to determine whether CMX001 is a substrate of human Organic Anion Transporter 1 (hOAT1) and hOAT3 using cell-based methods.

Cidofovir (CDV) is a polar, acyclic nucleoside phosphonates that is FDA-approved as Vistide® (cidofovir injection) for the treatment of cytomegalovirus retinitis. CMX001 has demonstrated increased potency in cell based assays relative to CDV and has proven effective in vivo in animals after oral administration. Importantly, no signs of nephrotoxicity have been observed in animal toxicology studies or in human clinical trials to date after oral administration of CMX001, a distinct advantage compared to CDV, which is known to accumulate in kidney proximal tubule cells through their selective uptake by organic anion transporter 1 (OAT1) and OAT3.

### Method

Cellular uptake. MDCK-II cells were grown on semi-permeable filters (1 μM, polyethylene terephthalate (PET), Millipore), and transiently transfected with hOAT1, hOAT3 or vector only using a proprietary technique (Optivia Biotechnology). Compound (CMX001 and CDV at 25 μM), with or without probenecid, an OAT inhibitor (100 μM), in the presence or absence of 20% human serum, were added to the basolateral side of the cell monolayer (n=4 replicates/condition). After a 5 min incubation period, drug solutions were removed from cells, and the cells were rinsed, extracted with 50% acetonitrile, and analyzed by LC/MS/MS. Net OAT-mediated uptake was determined from total uptake in OAT-expressing cells minus uptake in vector-treated control cells. Minimum established acceptance criteria for hOAT1 activity was >0.71 pmol/min/cm² for p-aminohippurate (PAH), with >70% inhibition in the presence of probenecid; and for hOAT3 activity was >0.62 pmol/min/cm² for estrone-3-sulfate, with >90% inhibition in the presence of probenecid. Statistical significance (p<0.05) of the cellular uptake of compounds in transfected vs. control, ±probenecid or ±20% serum was assessed using an unpaired t-test. Statistical analysis of multiple parameters was performed using analysis of variance (ANOVA).

LC/MS/MS. Cell extracts were analyzed (Integrated Analytical Solutions) for CMX001 using a Peeke Scientific Polymeric SDB (10×2 mm) column with an initial mobile phase of 90% water/8% acetonitrile/2% tetrahydrofuran (containing 0.1% (v/v) formic acid), held constant for 0.25 min, and then changed to 80% acetonitrile/20% tetrahydrofuran (containing 0.1% formic acid, v/v) over a 1.25 min linear gradient. The flow rate was 0.8 mL/min. Total ion chromatograms and MS-MS spectra of ions (m/z 562.4/261.9 and 570.5/269.9, respectively) were obtained using an API 3000 mass spectrometer using electrospray ionization (400° C.) in positive ion mode. For CDV, a Peeke Scientific HILIC SM (30×2.1 mm) column with an initial mobile phase of 95% acetonitrile/5% water (containing 0.1% (v/v) formic acid), held constant for 0.25 min, and then changed to 5% acetonitrile/95% water (containing 0.1% (v/v) formic acid)) over a 1.25 min linear gradient was used. The flow rate was 0.8 mL/min, and ions (m/z 280.2/86.0) were detected as described above. The flow rate was 0.8 mL/min, and ions (m/z 288.2/176.3) were detected as described above.

### Results

Net uptake of CMX001 (5 μM) was not enhanced in hOAT1 and hOAT3 expressing cells, nor was uptake decreased in the presence of OAT inhibitor probenecid. Net uptake of CDV (25 μM) was enhanced in hOAT1 expressing cells, and decreased in the presence of OAT inhibitor probenecid, but was not enhanced in hOAT3 expressing cells. In the presence of 20% serum, uptake of CMX001 in hOAT1-expressing cells and control cells was substantially reduced, indicating that passive uptake of both compounds is reduced by binding to serum protein.

TABLE 2
Effect of Serum on hOAT1-Mediated Uptake of CMX001

| | | | | Net |
|---|---|---|---|---|
| Compound | | Uptake | Uptake | Transporter |
| (Concentration) | Treatment | (Transporter) | (Control) | Uptake |
| CMX001 | Buffer | 15.3 ± 5.4 | 15.6 ± 4.3 | −0.3 ± 5.4 |
| (5 μM) | 20% serum | BLOQ | 0.6 | ND |
| CDV (25 μM) | Buffer | 117 ± 15 | 13.8 ± 5.5 | 104 ± 15 |
| | 20% serum | 116 ± 22 | 13.5 ± 8.0 | 102 ± 22 |

Values are mean ± std deviation in units, pmol/min/cm²;
BLOQ, below limit of quantitation;
ND, not determined.

CMX001 uptake was not enhanced in vitro in cells expressing hOAT1 or hOAT3 compared to control cells that do not express these transporters under the conditions used. In contrast, uptake of CDV was enhanced in vitro in cells expressing hOAT1, which confirms literature reports of efficient uptake of CDV by hOAT1. Despite demonstrated functional uptake of hOAT3 substrate estrone-3-sulfate, no hOAT3-mediated transport of CDV was observed. This result is consistent with the reported lower uptake efficiency (i.e., >100-fold lower Vmax/Km) for CDV, though some groups have observed enhanced in vitro uptake of CDV by hOAT3.

CMX001 is not a substrate for hOAT1 or hOAT3. These data combined with the lack of nephrotoxicity observed to date in animals and humans following oral administration of CMX001, suggest that CMX001 has a low potential to cause OAT-mediated nephrotoxicity, a known adverse event following administration of CDV.

What is claimed is:

1. A method of treating an orthopox virus infection or a disease associated with an orthopox virus in an immunocompromised human subject, comprising administering to the subject a pharmaceutical composition comprising a therapeutically effective amount of a compound of the structure: 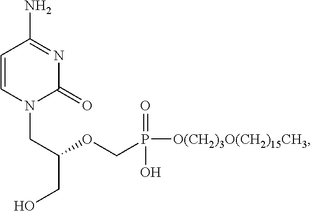
or a pharmaceutically acceptable salt thereof and a pharmaceutically acceptable carrier;
wherein the compound is administered once a week for three weeks in an amount of about 200 mg.

2. The method of claim 1, wherein the disease is an orthopox virus infection.

3. The method of claim 1, wherein the disease is a disease associated with an orthopox virus.

4. The method of claim 1, wherein the orthopox virus infection is variola virus.

5. The method of claim 1, wherein the orthopox virus infection is smallpox.

6. The method of claim 1, wherein the orthopox virus infection is vaccinia.

7. The method of claim 1, wherein the orthopox virus infection is monkeypox.

8. The method of claim 1, wherein the orthopox virus infection is molluscum contagiosum.
